(19) United States
(12) Patent Application Publication
(10) Pub. No.: US 2010/0080860 A1
(43) Pub. Date: Apr. 1, 2010

(54) Application of short-chain monocarboxylic acids for crop protection

(75) Inventors: Pilar García Agustín, Valencia (ES); VICTOR Flors Herrero, Castello De La Plana (ES); Carmen González Bosch, Burjassot (Valencia) (ES); Maria Dolores Real García, Burjassot (Valencia) (ES)

Correspondence Address: BERENBAUM WEINSHIENK PC, 370 17TH STREET, SUITE 4800, DENVER, CO, 80202, US

(73) Assignees: UNIVERSITAT JAUME I, Castelló De La Plana (ES); UNIVESITAT DE VALENCIA, València, (ES)

(21) Appl. No.: 11/993,775

(22) PCT Filed: Jun. 22, 2006
(86) PCT No.: PCT/ES06/70087

§ 371 Date: Jan. 8, 2008

(30) Foreign Application Priority Data
Jun. 23, 2005 (ES) P200501535

(51) Int. Cl. A61K 33/02; A61K 31/19; A61K 31/7004; A61K 31/185

(52) U.S. Cl. 424/719; 514/557; 514/23; 514/578

(57) ABSTRACT

The present invention relates to the agricultural use of hexanoic acid and monocarboxylic acids with similar chain structure having from 5 to 8 carbon atoms, their derivatives, their salts, and aqueous mixtures thereof containing modified hexoses and/or amines. These compounds are useful as plant growth stimulants, anti-senescents, and as resistance inducers to biotic and abiotic stresses. The invention also refers to the use of caproic (hexanoic) acid as an inhibitor of spore germination and mycelium growth of phytopathogenic fungi. The use of hexanoic acid as a fungicide in both pre- and post-harvest treatments is also suggested. The inducing activity of hexanoic acid on plant defenses against phytopathogenic fungi at non-phytotoxic concentrations has been shown.

The invention relates to the technical field of phytosanitary products. Particularly, the invention refers to the use of short-chain monocarboxylic acids or derivatives thereof for crop protection, and more particularly to their use as resistance inducers against different types of stress on plants in pre- and post-harvest treatments. The invention also refers to their use as biocidal agents.

### BACKGROUND OF THE INVENTION

Exogen applications of phytohormones, such as cytokinines, auxins, giberelins and ethylene show certain restrictions due to the high response variability observed and the different effects caused, depending on cultivation conditions (cf., Alexieva V., “Chemical structure, plant growth regulating activity of some naturally occurring and synthetic aliphatic amines”, 1994, Compt. Rend. Acad. Bula. Sci, vol. 47, pp. 779-782)). At the same time, on account of the multiplicity of physiological effects exerted on plants, these phytoregulators may elicit nutritional, flowering and growth disorders. Some phytohormones, such as synthetic cytokinines, because of their similarity to nitrogenated bases of nuclei acids, may induce physiological alterations (cf., Al-Khativ K, et al., “Use of growth regulators to control senescence of wheat at different temperatures during grain development”, 1985, Journal of Agricultural Food and Chemistry, vol. 33, pp. 866-870).

Thus, the researchers are making every effort in developing novel plant growth regulators for several years.

Early studies on the application of dicarboxylic acids on plants were conducted by Muñoz (cf., Muñoz, C. S., “Physiological alterations in corn Zea mays L. using monoesters from some low weight organic acids”, 1978, Graduate College ESAHE, Research Report, School Main Library) and Velichkov (cf., Velichkov, D. et al., “Effects of some aliphatic dicarboxylic acid esters on soybean Glycine max M. photosynthesis and transpiration”, 1989, Fiziolna. Rast. Sofia, vol. 15, pp. 21-26). These studies revealed that photosynthesis was stimulated in plants under foliar treatments with succinic acid monoester and adipic acid monoester, which produced an increase in biomass and a nutrient assimilation improvement.

As experimental data have being gathered, as those previously described, it has been made evident that they act on the basic mechanisms in plants. Thus, Stutte et al. (cf., Stutte et al., “Evolutions of carboxylic acids on soybean nutrients uptake, 1989, Research Report, University of Arkansas, pp. 3) demonstrated that there is a direct relationship between foliar application of trihydroxyglutaric acid and the increase in the concentration of malic acid/citric acid in roots and citric acid in stems. This ensures a higher assimilation rate of nutrients and water, and a larger transport via xylem.

It is remarkable that later studies show that hydroxyglutaric acid favoured the synthesis of polyalcohols, the increase in circulating polyalcohols, the radical development, and the photosynthetic processes.

Moreover, evidence that the application of amines may alter the phenolic content of leaves by acting on shikimic acid pathway has also been shown. Since this pathway is involved in plant defense mechanisms, such treatment may protect a plant against the potential attack of pathogens (Del Rio et al., “Effect of benzylaminopurine on the flavonones hesperidin, hesperitin 7-O-glucoside and purin in tangüelo Nova frutis, 1995, J. Agric. Food Chem., vol 43 (8), p. 2030-2034). Accumulation of phenolic compounds, isoflavonoids, and their precursors is a habitual response of plants to a fungal elicitor or to a pathogen attack.

Some compounds are known to act as inducers of plant natural defenses, for example, salicylic acid and its structural analogs benzothiadiazol (BTH) and isonicotinic acid (INA), as well as chitosans, or non-proteic β-aminobutyric aminoacid (BABA). Thus, as it is derivable from the art, the provision of compounds that can strengthen plant defenses against different stress conditions is still of great interest in the agriculture sector.

The effectiveness of these compounds varies among plant species and among monocotyledon and dicotyledon species. As currently known, the ability of BABA to induce resistance depends on abscisic acid (ABA)-mediated signalling pathway and chalose accumulation.

Apart from the compounds that may exert direct control on pathogen growth, the research for novel compounds to get the control of pathogen diseases by stimulating endogen defenses of plants is of great interest.

### DESCRIPTION OF THE INVENTION

The present invention refers to the agricultural use of hexanoic acid and monocarboxylic acids with similar chain structure having from 5 to 8 carbon atoms, their derivatives and their salts, and aqueous mixtures of them with modified hexoses and/or amines. The compounds of the invention are useful as plant growth stimulants, anti-senescents, and as resistance inducers to biotic and abiotic stresses in different plant species and, consequently, for crop protection. The invention also refers to the use of these compounds in pre- and post-harvest applications or as direct biocidal agents against bacteria, oomycetes, nematodes, fungi, viruses and insects. When applied at higher concentrations than those used in inducer treatments for plants, hexanoic acid shows a direct fungicidal effect.

An advantage of the agricultural use of the compounds of the present invention is that they have a wide-spectrum of action and are able to become effective in very distinct plant species, both horticultural and ornamental, and even woody species. When applied to the plant via radical, at low concentrations (0.6 mM), these compounds stimulate plant defense mechanisms.

Furthermore, the compounds of the invention are able to protect crops without involving an additional cost to the plant and without producing apparent effects in the absence of stress (this phenomenon is termed priming). In this respect, it has been shown that the capacity of inducing defenses in vegetables by the imitation of hormonal signals may involve a metabolic cost to the plant, since crop protection is usually accompanied by the induction of the so-called hypersensitive response (HR) (necrosis in the absence of pathogens) and even by the decrease in growth and/or production (cf., van Hulten et al, “Costs and benefits for priming for defense in Arabidopsis”, 2006, PNAS 103: 5602).

However, the use of the monocarboxylic acids according to this invention results in a suitable protective effect, since plant defenses are induced without any metabolic cost. Thus, the induction of unwanted responses, e.g., hypersensitivity, is prevented, and plant growth and/or production are not affected.

In addition, the researchers of the present invention have found that the inducer effect of short-chain monocarboxylic acids having from 5 to 8 carbon atoms is more potent than that obtained with dicarboxylic acids or amines. Consequently, by using lower concentrations of monocarboxylic acids, the same effect as that by other compounds described in the art is achieved, with the additional advantage that the use of lower concentrations prevents the crop (plant or fruit, etc.) from toxicity problems.

In accordance with the aim of the present invention, the use of short-chain monocarboxylic acids having from 5 to 8 carbon atoms refers to both the use of a single monocarboxylic acid and to a mixture thereof.

Illustrative and non-limitative examples of short-chain monocarboxylic acids having from 5 to 8 carbon atoms include pentanoic acid (C5), hexanoic acid (C6), heptanoic acid (C7) and octanoic acid (C8).

In the present invention, the term “biotic stress” refers to the damage caused by pathogens, such as fungi, oomycetes, bacteria, nematodes, viruses and insects.

In the present invention, the term “abiotic stress” refers to the damage caused by salinity, drought and nutrient deficiency.

In the present invention, the term “biocidal agent” refers to a substance that can kill a variety of organisms.

In a preferred embodiment, the present invention refers to the use of short-chain monocarboxylic acids having from 5 to 8 carbon atoms or their derivatives, or an aqueous mixture thereof with modified hexoses and/or amines for crop protection.

In another preferred embodiment, the present invention refers to the use of at least one short-chain linear monocarboxylic acid having from 5 to 8 carbon atoms, their derivatives and their salts, or an aqueous mixture thereof with modified hexoses and/or amines.

In yet another preferred embodiment, the present invention refers to the use of short-chain monocarboxylic acids having from 5 to 8 carbon atoms, or an aqueous mixture thereof with amines for crop protection.

In yet another embodiment of the present invention, the amines of the aqueous mixture are selected from the group consisting of: ammonia, 1,3-diaminopropane, furfurylamine, allantoin, putrescine, spermidine, spermine, α-aminoacids and a mixture thereof.

In yet another embodiment of the present invention, the salts of monocarboxylic acids are alkaline or alkaline earth metal salts. Preferably, the salts of monocarboxylic acids are potassium salts.

As a matter of fact, the inventors of the present invention observed that by using a salt or a mixture of alkaline or alkaline earth salts of 5-8 carbon atom monocarboxylic acids, an improved effect was achieved (see Example 3).

The present invention refers to the use of caproic (hexanoic) acid or an alkaline or alkaline earth salt thereof, as an inhibitor of both spore germination and mycelium growth of phytopathogen fungi. Another suggested use is as a fungicide for pre- and post-harvest treatments, as well as an inducer of plant defenses against phytopathogen fungi and certain abiotic stresses without resulting in phytotoxic effects at the concentrations used.

This dual effect on the fungus (fungicide) and on the plant defenses (inducer) makes this product a very attractive candidate in combating infections caused by pathogenic fungi. This product also proves to be effective against other pathogen types, such as bacteria and viruses.

In a preferred embodiment, the concentration of hexanoic acid ranges from 3 to 16 mM for showing fungicidal effect.

In another preferred embodiment, hexanoic acid is applied in solution form at pH 3-6.

In yet another preferred embodiment, the salt of hexanoic acid is the potassium salt.

The use of hexanoic acid for inducing resistance to plants has the following advantages:
a) In either hydroponic or soil cultivations, the root application of hexanoic acid significantly enhances the protection of tomato plants against B. cinerea. This inducer effect is dependent on concentration, at a range from 0.6 to 30 mM, showing a predominantly inducer effect at 0.6 mM, since a direct fungicidal effect is already present at 3 mM (see Tables 1 and 2);
b) The protective level is similar to that of well-characterized inducers such as β-aminobutyric acid (BABA).
c) It is effective on fungi both in vitro and by spray application to plants.
d) Hexanoic acid significantly decreases germination starting from 3 mM concentrations and entirely inhibits germination at 16 mM concentrations. This proves that hexanoic acid exerts a fungicidal effect by affecting the early stages of the germination process, since the germ tube development is prevented. Likewise, the effect of heptanoic acid was found to be the same at the same concentration range than the hexanoic acid.
e) The inhibitor effect of hexanoic acid is maintained after inoculating spores treated with the minimum fungicidal concentration (hereinafter abbreviated as MFC) of hexanoic acid in fruits and tomato leaves. This effect is an irreversible fungicidal effect, since spores do not recover their germinating capacity after removing the treatment by spore washing.
f) Hexanoic acid inhibits mycelium development in the already germinated spores.

The dual effect shown by hexanoic acid, on both spore germination and hypha development, is particularly interesting since other known fungicides affect preferentially one of the processes. In fact, spore germination constitutes the most sensitive stage in plant development to inhibition by the great majority of antimicrobial compounds, for example, the class of strobirulin-related fungicides, which are rather ineffective as inhibitors of mycelium growth. However, other compounds strongly inhibit mycelium growth without affecting spore germination, for example, compounds affecting the microtubules, such as carbendazim and N-phenylcarbamates, which inhibit nuclear division, as well as ergosterol biosynthesis inhibitors.

Throughout the description and claims the word “comprise” and variations of the word, such as “comprising”, is not intended to exclude other technical features, additives, components, or steps. Additional objects, advantages and features of the invention will become apparent to those skilled in the art upon examination of the description or may be learned by practice of the invention. The following examples and drawings are provided by way of illustration, and is not intended to be limiting of the present invention.

### EXAMPLES OF THE INVENTION

The results obtained from the use of hexanoic acid (also termed caproic acid) show that it is effective in horticultural plants such as tomato plant, and in model plants such as Arabidopsis thaliana. This suggests that hexanoic acid may have a wide-spectrum of action and is able to become effective in very distinct plant species, both horticultural and ornamental, and even woody species.

The root application of hexanoic acid in tomato plants enhances resistance to the neurotrophic fungus Botrytis cinerea. This pathogen is responsible for important losses in tomato crops, which attacks both young plantules and different tissues (leaves, stems and fruits).

Studies performed with Arabidopsis thaliana showed that hexanoic acid induced resistance to different pathogens, such as the necrotrophic fungus Alternaria brassicicola, biotrophic oomicete Peronospora parasitica and bacterium Pseudomonas syringe.

Although the mechanism of action of hexanoic acid has not yet been elucidated, there are some preliminary data available from comparative tests with other well characterized inducers, such as β-aminobutyric acid. The inducing effect of this compound against necrotrophs is, in part, due to a rapid callose deposition at the penetration sites of fungi. Furthermore, mutants of Arabidopsis, which appear insensitive to defense induction by other chemicals, are similarly insensitive to the resistance induced by hexanoic acid. These results demonstrate that this compound applied to plants by root application, does not preferentially act as a fungicide with direct effects on pathogens but it stimulates defense mechanisms which, for the time being, are unknown.

Other studies performed with fungi in in vitro culture have shown that hexanoic acid, at higher concentrations than those used in plant treatments, may have a direct fungicidal effect. Testing data aim at an inhibiting effect on spore germination, as well as hypha development.

Preliminary studies were also performed on the application of hexanoic acid for post-harvest treatment. Current studies show that combined inoculation of hexanoic acid and Botrytis cinerea spores into tomato fruits produces a significant decrease in the infection rate of treated fruits, and pathogen inhibition is entirely achieved at higher doses.

It is of particular interest to emphasize that the use of other similarly structured carboxylic acids according to the present invention has shown that these compounds, when combined in aqueous solutions with either modified hexoses or amines, have a stimulant effect on plant growth and reduce fungal infections. Likewise, when these compounds are combined by ester and/or amide bonds with sugars and amines, respectively, a similar effect is produced, being enhanced the resistance of plants to biotic and abiotic stress is enhanced.

### Example 1

### Induction of Resistance to Biotic and Abiotic Stress Studies on Tomato Plants

a) Effect on Necrotrophs: B. cinerea

Experiments were performed with 4-week-old tomato plants (Cv. Ailsa Craig) in hydroponic cultivation. Before initiation of treatment, the plants were maintained in hydroponic cultivation for 1 week for habituation under such cultivation conditions. Treatment comprised the addition of hexanoic acid to a nutrient solution (Hoagland solution) at the following concentrations: 0.06 mM, 0.6 mM, 3 mM, 6 mM, 16 mM and 20 mM, pH was adjusted to 6. After 48 hours of treatment, the leaflets of the third and fourth true leaves were inoculated with 5 μl of a suspension containing 10⁶ conidia B. cinerea/ml. Spores had previously been incubated in Gamborg medium (Duchefa Biochemie, The Netherlands) supplemented with sucrose and phosphate, over a period of 2 h. Spores were inoculated in this medium. For each treatment, 10 plants were used, which were kept under humidity- and temperature-controlled conditions (80% RH, 21° C.). Sampling was performed at 48, 72, 96 and 120 hours after inoculation.

The results obtained are as follow:

TABLE 1
Inducing effect of hexanoic acid root application, at different
concentrations in hydroponic cultivation, on the protection of tomato
plants against B. cinerea:

| | 0.06 mM | 0.6 mM | 3 mM | 6 mM | 16 mM | 20 mM |
|---|---|---|---|---|---|---|
| % inhibition | 35.57 | 48.90 | 64.06 | 61.98 | 63.52 | 53.60 |
| of infection | | | | | | |
| versus | | | | | | |
| control | | | | | | |

Control: water

Thus, hexanoic acid in hydroponic cultivation has shown to protect tomato plants against B. cinerea, very likely acting as an inducer at a concentration of 0.6 mM, since a fungicidal effect is already present from a 3 mM concentration.

### Hexanoic Acid Application in Soil Cultivation

Plants were cultivated in soil and the solutions of hexanoic acid, at different concentrations, were prepared and applied in the same manner as that described above. Control was water.

The root application of hexanoic acid in soil is also effective by enhancing the protection of tomato plants against B. cinerea. The inducing effect is accomplished at low concentrations (0.6 mM) as shown in Table 2.

TABLE 2
Inducing effect of hexanoic acid root application in soil cultivation
on the protection of tomato plants against B. cinerea

| | 0.06 mM | 0.6 mM |
|---|---|---|
| % inhibition of infection versus control | 60.15 | 49.77 |

Control: water

b) Effect on Arabidopsis thaliana

Experiments were performed with 5-week-old Arabidopsis plants cultivated in soil. Two weeks after germination, the plants were transplanted into single pots and kept at a temperature of 20° C. day/18° C. night and 8.5 h light cycle for 24 h and 60% relative humidity (RH).

The plants were treated with different concentrations of hexanoic acid (0.6 mM, 0.8 mM and 1 mM) and inoculated with 6 μl of 2.5×10⁴ spores/mL Botrytis cinerea and 2×10⁶ spores/mL Alternaria brassicicola.

Sampling was performed at 48, 72, 96 and 120 hours after inoculation.

TABLE 3

| | | |
|---|---|---|
| 0.6 mM | 0.8 mM | 1 mM |

A)

| | | | |
|---|---|---|---|
| % inhibition of infection versus control | 10 | 28 | 38 |

B)

| | | | |
|---|---|---|---|
| Time after inoculation | 48 | 72 | 96 |
| % inhibition of infection versus control | 54.5 | 40.5 | 6.3 |

A) Inducing effect of hexanoic acid root application in soil cultivation on the protection of Arabidopsis plants against B. cinerea.
B) Effect of hexanoic acid root application (0.8 mM) in soil cultivation, at different times after inoculation, on the protection of Arabidopsis plants against A. Brassicicola

Thus, hexanoic acid root application has shown to enhance defense induction of Arabidopsis plants against a variety of phytopatogens. Among these, the necrotrophic fungi Botrytis cinerea and Alternaria brassicicola are emphasized (Table 3). That proves the inducing effect of hexanoic acid at low concentrations.

### Example 2

### Direct Antimicrobial Effect of Hexanoic Acid and Heptanoic Acid

Test mixture contained 4×10⁶ conidia/ml PDB (potatodextrose broth, Difco, Detroit, Mich., USA). Treatments (hexanoic acid and heptanoic acid) were added to PDB from stock solutions in order to obtain the different concentrations to be used (0.06 to 30 mM). Medium pH was adjusted to 3.6-5.5 using HCl or NaOH. One ml of each mixture was dispensed into sterile 24-well microplates and then incubated at 20° C. under gentle stirring. Percentage of germinated spores was calculated after 20 h incubation by staining with 0.1% lacto-fucshin (1:1) followed by microscopic observation of 100 spores. Spores were considered to be germinated when the length of germ tube was equal to or greater than spore. For assessment of efficacy in long-term treatments, test culture was left for a 7-day incubation period under previously described conditions, and mycelium development was assessed as follows: 3 ml of each culture were vacuum-filtered through pre-weighed nitrocellulose filters (Millipore, ref. HAWP02500). The filters were dried at 85° C. until weight was constant at room temperature.

For measurement of treatment effects on mycelium growth, 9×10⁶ spores were inoculated to a final volume of 150 ml PDB. After 20 h incubation, spores were examined for germination by microscopic analysis, then treatments were added, and pH was adjusted to 5.5. Cultures were incubated at 20° C. under gentle stirring. After 96 h, mycelium growth was measured by determining dry weight, as described herein. Minimum inhibitory concentration (MIC), which is the lowest concentration of test compounds that did not produce spore germination or visible mycelium growth following 20 h incubation (germination period of B. cinerea spores under the conditions used herein), was then determined. After MIC determination, minimum fungicidal concentration (MFC) was measured as follows. After 20 h incubation, an aliquot of the cultures, in which no growth had been detected, was taken, then washed three times and added to a fresh medium without added treatment. After 20 h incubation, under previously described conditions, the germination percentage was estimated. MFC is the minimum concentration of test compound at which microorganisms are not recovered.

Results:

TABLE 4
Effect of spray application of hexanoic acid at different
concentrations on tomato plants against B. cinerea

| | 3 mM | 16 mM | 20 mM |
|---|---|---|---|
| % inhibition of invention versus control | 7.63 | 66.85 | 81.47 |

The spray application to tomato plants has enabled to show the efficacy of hexanoic acid as an antimicrobial treatment. The application of hexanoic acid at the MFC (16 mM) reduced fungal necrosis by 67%, the effect being even higher at 20 mM with 81% reduction (Table 4)

Preliminary results have evidenced that the application of hexanoic acid may also have a curative effect in plants already inoculated with the fungus B. cinerea, since its application to these plants restrains the progress of necrosis.

Heptanoic acid was also shown to be effective as a spore germination inhibitor from a concentration of 6 mM. In such a case, like hexanoic acid, heptanoic acid entirely prevents germ tube development, thus indicating that it acts during the early stages of germination.

### Example 3

### Spray Treatment of Tomato Plants with Hexanoic Acid Potassium Salt (Cv Ailsa Craig)

Hexanoic acid potassium salt was provided by preparing a mole-to-mole solution of hexanoic acid with K₂CO₃. A concentrated solution was prepared, adjusted to pH 6, and then dilutions were performed in order to provide in-use concentrations, namely, 20 mM, 10 mM, 6 mM and 3 mM.

The plants were treated with the formed salt by spray application and then left to dry for 30 minutes. Afterwards, treated leaves, which were previously incubated for 2 h in Gamborg medium supplemented with sucrose and phosphate, were inoculated with 5 μl of a suspension containing 10⁶ spores/ml of B. cinerea, and then stored in a high-humidity chamber at 25° C. for 72 h.

The infection diameter was measured and, finally, the germination percentage was determined.

The results obtained are as follow:

| | 3M | 6 mM | 10 mM | 20 mM |
|---|---|---|---|---|
| % inhibition of infection | 49.8 | 52.1 | 61.3 | 80.3 |
| versus control | | | | |

Results show that the most effective dose of the potassium salt is 20 mM. There was 80% inhibition of fungal infection. When the dose was applied at 3 mM, an inhibition of about 50% was also observed, which was not observed when the hexanoic acid was applied alone.

1. A method of use of one or more short-chain monocarboxylic acids having from 5 to 8 carbon atoms, their salts, or an aqueous mixture thereof with modified hexoses and/or amines as resistance inducers to biotic and abiotic stresses for crop protection prior to the infection by a pathogen, including applying the short-chain monocarboxylic acid, salt or mixture to a crop plant.

2. A method of use according to claim 1, of short-chain monocarboxylic acids having from 5 to 8 carbon atoms, or aqueous mixture thereof with modified hexoses and/or amines.

3. A method of use according to claim 1, of at least one short-chain linear monocarboxylic acid having from 5 to 8 carbon atoms, their salts, or an aqueous mixture thereof with modified hexoses amines.

4. A method of use according to claim 1, of short-chain monocarboxylic acids having from 5 to 8 carbon atoms, or an aqueous mixture thereof containing amines.

5. A method of use according to claim 4, wherein the amines of the aqueous mixture are selected from the group consisting of: ammonia, 1,3-diaminopropane, furfurylamine, allantoin, putrescine, spermidine, spermine, α-aminoacids and a mixture thereof.

6. A method of use according to claim 1, wherein the salts of monocarboxylic acids are alkaline or alkaline earth metal salts.

7. A method of use according to claim 6, wherein the salts of monocarboxylic acids are potassium salts.

8. A method of use according to claim 1, wherein the monocarboxylic acid is hexanoic acid or an alkaline or alkaline earth metal salt thereof.

9. A method of use according to claim 8, wherein the salt is the potassium salt of the hexanoic acid.

10. A method of use of claim 8, wherein the hexanoic acid is used at a concentration which does not result in phytotoxic effects.

11. (canceled)

12. A method of use according to claim 8, wherein the hexanoic acid is applied in solution form at a pH value ranging from 3 to 6.

13. (canceled)

14. (canceled)

15. (canceled)

16. (canceled)

17. (canceled)

18. (canceled)

19. A method of use of at least one short-chain monocarboxylic acids having from 5 to 8 carbon atoms, their salts, or an aqueous mixture thereof with modified hexoses or amines as resistance inducers to biotic and abiotic stresses for crop protection prior to the infection by a pathogen, including applying the short-chain monocarboxylic acid, salt or mixture to a crop plant at a concentration ranging from about 0.6 mM to about 3 mM.

20. A method of use according to claim 19, wherein the concentration is in a range from about 0.6 mM to about 1 mM.

21. A method of use according to claim 19, of short-chain monocarboxylic acids having from 5 to 8 carbon atoms, or an aqueous mixture thereof with modified hexoses and/or amines.

22. A method of use according to claim 19, wherein the amines of the aqueous mixture are selected from the group consisting of: ammonia, 1,3-diaminopropane, furfurylamine, allantoin, putrescine, spermidine, spermine, α-aminoacids and a mixture thereof.

23. A method of use according to claim 19, wherein the salts of monocarboxylic acids are alkaline or alkaline earth metal salts.

24. A method of use according to claim 23, wherein the salts of monocarboxylic acids are potassium salts.

25. A method of use according to claim 19, wherein the monocarboxylic acid is hexanoic acid or an alkaline or alkaline earth metal salt thereof.

26. A method of use according to claim 25, wherein the salt is the potassium salt of the hexanoic acid.

27. A method of use according to claim 25, wherein the hexanoic acid is applied in solution form at a pH value ranging from 3 to 6.
